(19) United States
(12) Patent Application Publication
(10) Pub. No.: US 2017/0165050 A1
(43) Pub. Date: Jun. 15, 2017

(54) ARTIFICIAL EYES AND MANUFACTURE THEREOF

(71) Applicant: The Manchester Metropolitan University, Manchester (GB)

(72) Inventors: Thomas George Fripp, Sheffield (GB); Lewis Green, Sheffield (GB); Lesley Elizabeth Gill, Manchester (GB)

(21) Appl. No.: 15/118,436

(22) PCT Filed: Feb. 11, 2014
(86) PCT No.: PCT/GB2014/050396

§ 371 Date: Aug. 11, 2016

(51) Int. Cl. A61F 2/14; B29C 45/00; B29C 45/14; B29D 11/02

(52) U.S. Cl. CPC A61F 2/141; B29D 11/02; B29C 45/0001; B29C 45/14065; B29C 45/14819; B29L 2011/0008

(57) ABSTRACT

A method of manufacturing an artificial eye for fitting as a whole or partial replacement of a patient's original eye. This is done by providing an image of an iris on a substrate (100, 300) comprising at least a frontal region of an artificial eye, providing a support (108, 308) for the substrate, positioning the substrate and support in a mould (122, 322), and encapsulating the substrate within a mould material (136, 336).

### FIELD OF THE INVENTION

The present invention relates to artificial eyes (ocular prostheses) and methods of manufacture thereof.

### BACKGROUND OF THE INVENTION

Artificial eyes have been prepared for patients whose eye(s) have been damaged due to injury or disease for several centuries. However, the techniques used remain skilled and labour intensive. Commonly, the prosthesis is made from acrylic plastics such as polymethylmethacrylate (PMMA) and this is encapsulated. Prior to encapsulation, maxillofacial prosthetists and ocularists simulate the colour of the iris and sclera using individual hand-painting techniques with the patient present (or from an image of the patient's eye). A variety of artists media are used which are applied by pencils, crayons, cotton or a brush. This technique requires inherent artistic ability and is time consuming and expensive. The result is dependent upon operator ability and experience.

To encapsulate the artificial eye a two stage moulding process is often used so as to ensure full encapsulation of the artificial eye. This is a time consuming process.

The present invention seeks to overcome or at least mitigate the problems of the prior art.

### SUMMARY OF THE INVENTION

A first aspect of the present invention provides a method of manufacturing an artificial eye for fitting as a whole or partial replacement of a patient's original eye, the method comprising the steps of:
a) providing an image of an iris on a substrate comprising at least a frontal region of an artificial eye;
b) providing a support for the substrate;
c) positioning the substrate and support in a mould; and
d) encapsulating the substrate within a mould material.

Preferably the support is a pin and/or the support may be provided on an internal side of the eye.

Provision of the support anchors the substrate in position in the tool which ensures that the substrate does not move during the encapsulation/moulding process. This means that the artificial eye can be formed with improved accuracy.

The support enables the substrate to be spaced from the mould so as to permit mould material to surround the substrate.

In the present application reference to a frontal (outer) and inner side of the substrate and/or artificial eye refers to the outer and inner sides when the artificial eye is in use. i.e. the frontal side is the side visible during use.

The support may be provided as a separate component to the substrate.

The method may comprise the step of providing a support that is made from a similar material as the mould material, for example the material may have the same of similar melting temperature or support may be made from the same material as the mould material. Advantageously, when the support is selected to be a material suitable for encapsulation, after the encapsulation/moulding process, the substrate is fully encapsulated in a mould material.

The support may be manufactured from a polymethylmethacrylate (PMMA) or acrylic. The support can made from a black opaque material so as to be used as the pupil of the eye. Alternatively the pupil may be a separate component from the support, or may be printed on the substrate as part of a 3D printing (additive manufacture) process. The mould material may be polymethylmethacrylate (PMMA) or acrylic and is preferably clear.

The mould may comprise a locator, for example a hole or surface. The support may engage the locator, for example the support may be inserted into the hole.

The method may comprise the step of positioning a head of the pin or other support on or through to an outer side of the substrate, e.g. to form the pupil of the artificial eye. The support may be a dark colour, e.g. black to form a pupil of the artificial eye. The material of the support may be selected such that the colour of the pupil does not fade substantially over time.

The method may comprise the step of providing a hole in the substrate and positioning the support through the hole of the substrate. The method may comprise the step of providing a recess in the substrate circumferentially around the hole for seating a head of the support.

The method may comprise the step of pushing the support from an outer side of the substrate through the hole towards the inner side of the substrate. A push fit provides a simplified construction method. The method may comprise the step of providing arms on the support positioned and arranged so as to permit the support to be pushed through the hole in the substrate and once in position anchor the support to the substrate.

The method may comprise the step of removing an end of the support once the substrate is encapsulated. For example, the end of the support may be snapped off once the substrate is encapsulated. Alternatively the end of the support may be cut off or melted off. Once the end of the support is removed, the region where the end of the support was located may be polished.

The method may comprise the step of providing a pin having a splined cross section. The splined cross section can improve location of the pin (and therefore the substrate) with respect to the mould. For example, the cross section of the pin may be cross-shaped.

The method may comprise the step of providing a disc along on the length of the support. The disc may be provided at a position to correspond with a depth of the mould once the substrate is encapsulated. The provision of such a disc eases removal of an end of the support. In particular, when the cross section is splined the disc can ease snapping or cutting off of an end of the support.

The frontal region of the substrate may be non-planar, preferably dome-shaped.

An orientation feature may be provided to orientate the substrate with respect to the support. A further orientation feature may be provided to orientate the support with respect to the mould. This arrangement may be particularly beneficial for angular orientation, if the above components require a particular angular position relative to each other. One example of this is the iris being off-centre from an apex of the substrate.

The support may be arranged to support substantially all an external or an internal face of the substrate.

The encapsulation step may include providing an unhardened mould material within a cavity of the mould, closing the mould, and hardening the mould material. This may be done by a heat and/or pressure curing process, e.g. with a two-part monomer and polymer mixture. In its unhardened form the mould material is preferably putty or gel-like so will conform to the voids in the mould.

The method may comprise the step of acquiring a digital image of an iris and transferring said digital image to the substrate. Such a step further reduces the cost of production and increases the effectiveness of an artificial eye compared to alternative to traditional manufacturing methods.

In such embodiments, the method may comprise the step of overlaying the image onto a 3D CAD model of an artificial eye. The image may be transferred to a substrate by a 3D printer.

Alternatively, the image may be printed onto a transfer material. For example, the transfer material may be a dye sublimation film and the image may be printed using dye sublimation ink.

The image of the iris may be colour corrected. For example, the image of the iris may be colour corrected to correct for the lighting conditions under which the image was acquired. Additionally or alternatively, the image of the iris may be colour corrected for the colour characteristics of a printer used to print the image.

The method may comprise a step of artificially adding lines representative of veins to a sclera portion of the image.

The method may comprise a step of scaling the image to an appropriate size. The image may be scaled to account for the optical effects of a subsequent encapsulation process. The image may be scaled to ultimately appear smaller than an iris of a patient's other original eye.

The image may be transferred to the substrate as an inherent part of the forming of the substrate in a 3D printer. The 3D printer may utilise powder and an adhesive binding system built up layer on layer to form the substrate. The powder may be a silica powder. The powder may be a substantially white powder.

The printed artificial eye may have a low viscosity bonding and strengthening agent applied thereto or infiltrated therewith. In some embodiments this may be a cyanoacrylate. In other embodiments it may be an acrylic. The artificial eye may be immersed in the bonding agent. Acrylic is preferred as it as a lesser tendency to absorb moisture compared to other material. The printed artificial eye may be cured by heat after infiltration.

The printed image may be transferred to the substrate from the transfer material using a vacuum transfer apparatus. A plurality of substrates may simultaneously have the printed image transferred thereto in the apparatus and a support grid may be provided to support the transfer material.

The method may comprise a step of applying an adhesion promoter to the substrate.

The method a may comprise a step of applying a fixing lacquer to the printed blank.

The blank may be a bespoke blank formed utilising a known blank manufacturing technique.

A second aspect of the present invention provides a pre-encapsulated or pre-moulded artificial eye comprising:
a substrate having at least a frontal region of an artificial eye;
an image of an iris positioned on an outer side of the substrate; and
a support for the substrate to suitably position the pre-moulded eye with a mould.

A third aspect of the present invention provides an artificial eye comprising:
a substrate layer having an image of an iris formed thereon;
a support located at an inner side of the substrate, and arranged to orientate the substrate with respect to the support;
transparent mould material arranged to encapsulate the substrate in conjunction with the support.

The artificial eye may be for fitting as a whole or partial replacement of a patient's original eye. The artificial eye may comprise a powder material bound together by a binder to form a shaped solid substrate. The binder may be selectively coloured in at least in a region thereof to simulate at least an iris portion of an eye.

The substrate may be non-planar, preferably dome-shaped, at least in the region that is selectively coloured. An iris portion of the substrate may be flat and/or recessed from the surface of the remainder of the surface. The iris portion may be off-centre from an apex of the dome. This may be advantageous for subsequent trimming operating that may occur to fit the competed artificial eye to a patient.

The support may be arranged to orientate the support with respect to a mould.

The support may made from a similar material to the mould material, preferably the same material.

The support may have a head and the head may be positioned to be visible on a frontal side of the substrate e.g. to form a pupil of the artificial eye.

The substrate may be formed at least partially from a 3D printed or additive manufactured material.

The substrate may be a shell-like structure. The binder may be selectively coloured on the concave and convex surfaces of the structure, or through a pre-determined depth of the substrate.

Dye-sublimated ink may be applied to simulate at least an iris of an eye.

The substrate may be a bespoke blank shaped to conform to a particular patient's eye socket. The colouring or ink may simulate a sclera portion of the eye.

A further aspect of the present invention provides a substrate for forming an artificial eye, the substrate being formed from an additive manufactured powder and printed during the additive manufacturing process with a representation of an eye feature, including at least one of an iris, a pupil or sclera, the substrate being infiltrated with an acrylic material.

Embodiments of the present invention will now be described in detail with reference to the accompanying drawings in which:

### BRIEF DESCRIPTION OF THE DRAWINGS

FIG. 1 is a perspective view of a substrate used in an embodiment of the present invention;

FIG. 2 is a cross-sectioned side view of the substrate of FIG. 1;

FIG. 3 is a perspective view of the substrate of FIG. 1;

FIG. 4 is a perspective view of a pin used in an embodiment of the present invention;

FIG. 5 is a sectioned view of the pin of FIG. 4 assembled with the substrate of FIG. 1;

FIG. 6 is a perspective view of the assembly of FIG. 5;

FIG. 7 is an end view of the assembly of FIG. 5;

FIG. 8 is a sectioned view of the assembly of FIG. 5 being positioned in a mould;

FIG. 9 is a perspective view of the assembly and mould of FIG. 8;

FIG. 10 is a further perspective view of the assembly and mould of FIG. 8;

FIG. 11 is a sectioned view of the assembly and mould of FIG. 8 at the end of a moulding process;

FIG. 12 shows a sectioned view of the assembly of FIG. 8 encapsulated in a moulding material;

FIG. 13 shows a side view of the encapsulated assembly shown in FIG. 12;

FIG. 14 shows a partially sectioned view of the pin of the encapsulated assembly being snapped off so as to form an artificial eye according to an embodiment of the invention;

FIG. 15 shows a fully sectioned side view of the pin and artificial eye of FIG. 14;

FIG. 16 is a flow chart illustrating a manufacturing method according to an embodiment of the invention; and

FIG. 17 is a flow chart illustrating two methods of manufacture according to an embodiment of the invention;

FIG. 18 is a digital image that is incomplete;

FIG. 19 is a digital image that has been manipulated and is ready for subsequent use;

FIG. 20 is a perspective view of a substrate used in another embodiment of the present invention;

FIG. 21 is a cross-sectioned side view of the substrate of FIG. 20;

FIGS. 22 and 23 are perspective views of a support used in an embodiment of the present invention;

FIG. 24 is a sectioned view of the support of FIGS. 22 and 23 assembled with the substrate of FIG. 20;

FIG. 25 is an exploded sectioned view of the assembly of FIG. 25 being positioned in a mould;

FIG. 26 is an isometric view of the mould of FIG. 25;

FIGS. 27 and 28 show a sectioned and perspective views of the assembly of FIG. 25 encapsulated in a moulding material;

FIG. 29 is a sectioned view of a variant of the substrate and support; and

FIG. 30 is a flow chart illustrating the steps of a manufacturing method for the components illustrated in FIGS. 20 to 29.

### DETAILED DESCRIPTION OF EMBODIMENT(S)

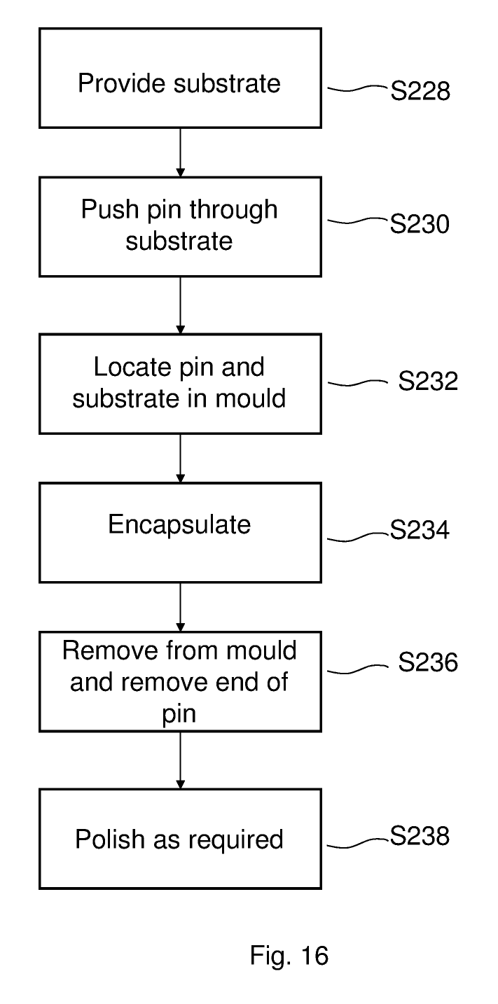

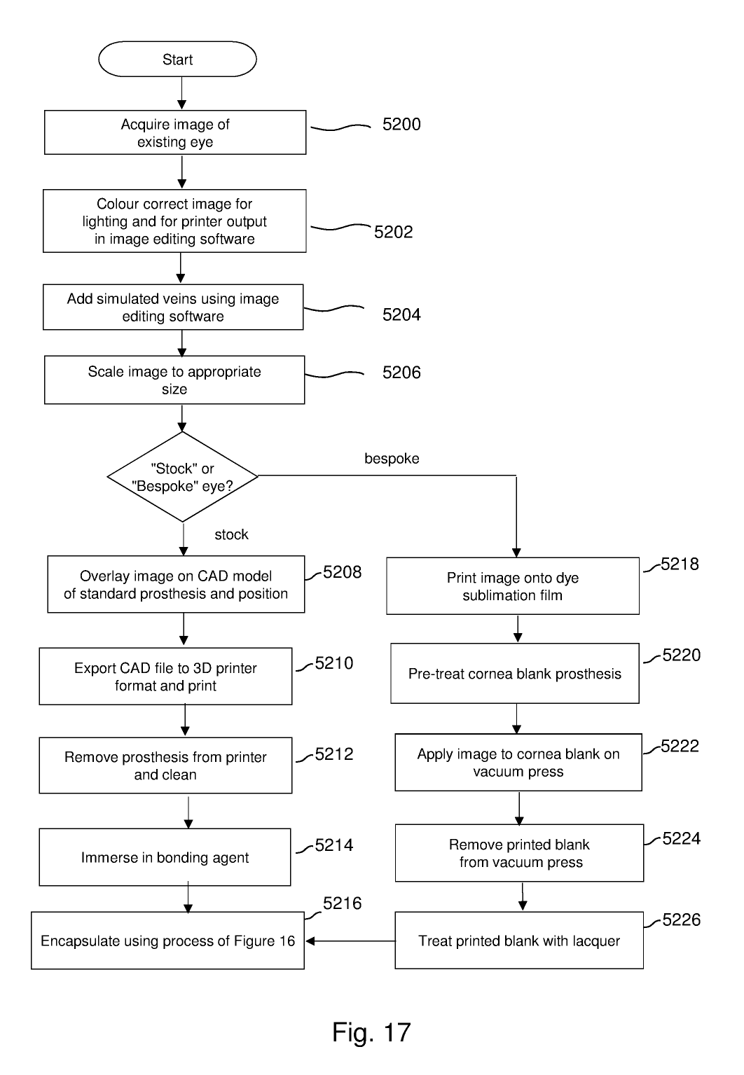

Referring to FIG. 16 a method of manufacturing an artificial eye according to an embodiment of the present invention is illustrated.

The first step of the method comprises providing a substrate. The substrate can be formed via known methods, or alternatively, as discussed below the substrate may be formed using a method of 3D printing or dye sublimation.

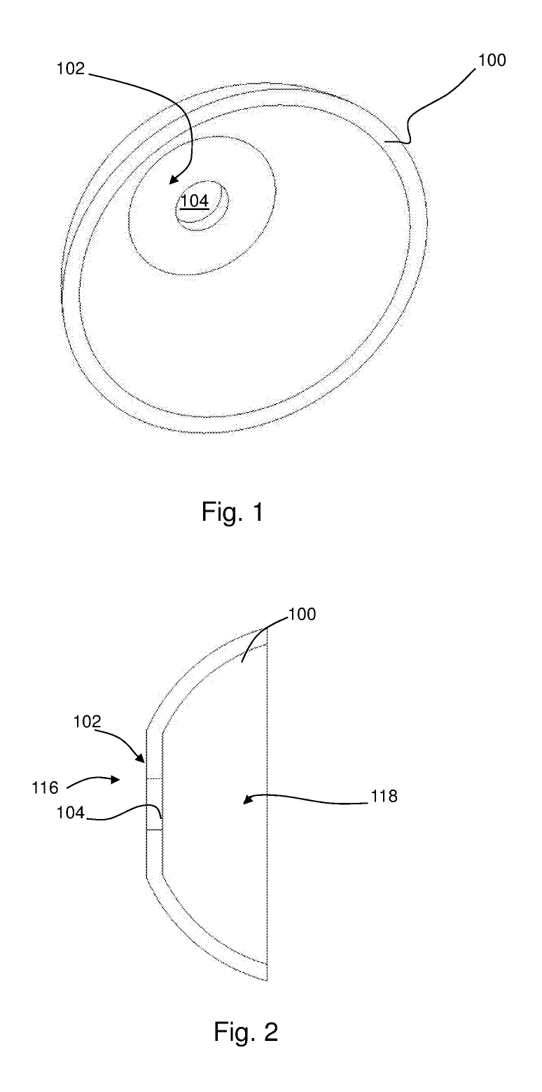

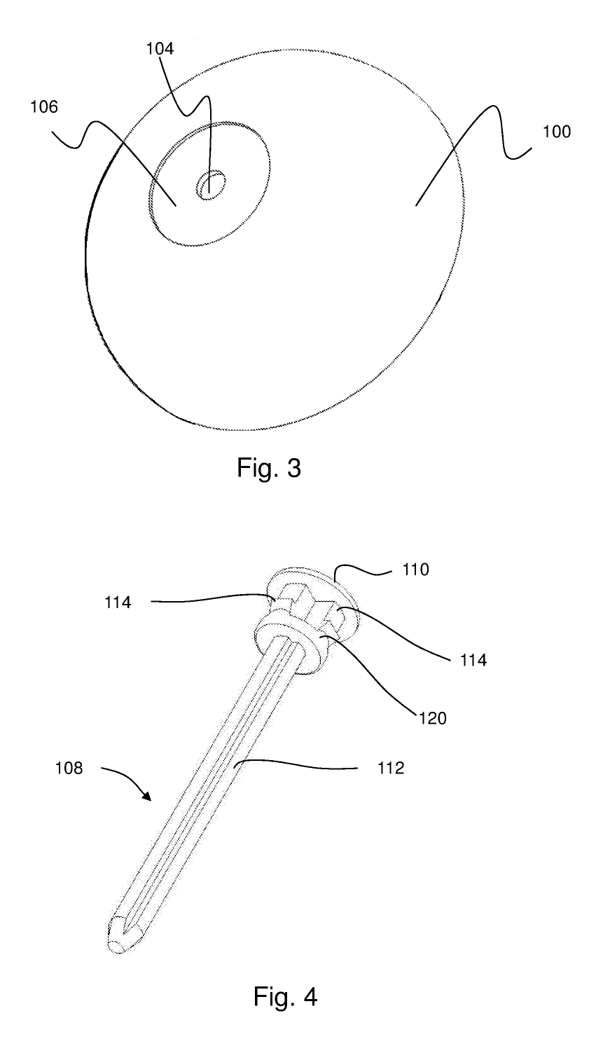

A substrate 100 is shown in FIGS. 1 and 2. In this embodiment the substrate has a convex shape, specifically a hollow domed shape. An apex 102 of the dome is flattened. A hole 104 is formed in the apex 102 of the substrate 100 and extends through the thickness of the substrate. In some embodiments, as shown in FIG. 3, a circular recess 106 may be formed around the hole 104. In this embodiment, the substrate is approximately 1 mm thick.

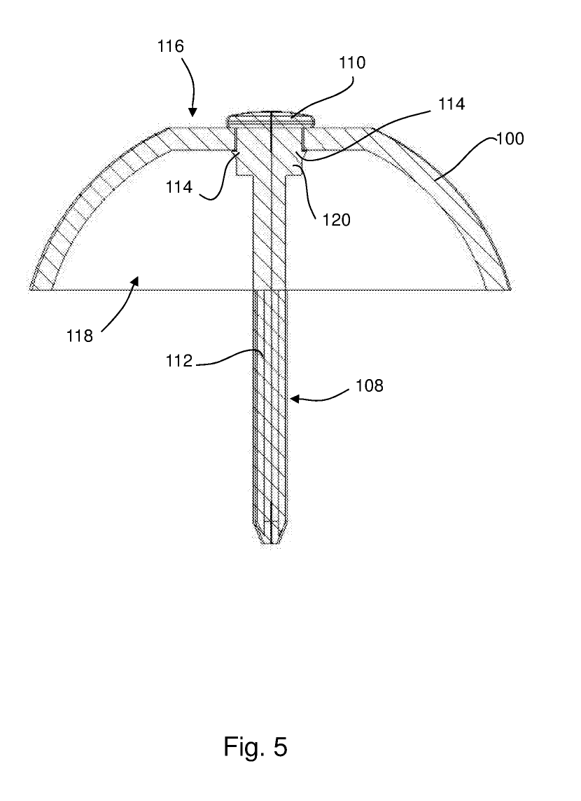

In a next step S230 a support in the form of a pin is pushed through the hole in the substrate. A pin 108 used in one embodiment is shown in FIG. 3. The pin has a head 110 which when correctly pushed through the substrate 100 will be seated in the recess 106. The head 110 may be flat or be domed (as illustrated in FIG. 5). The position of the head enables the head to form a pupil of the artificial eye. As such, it is preferred that the head 110 (and also the remainder of the pin 108 for ease of manufacture) is black in colour.

A longitudinal body 112 extends from the head 110 to an end of the pin 108. In this embodiment, the longitudinal body has a splined cross section, which in this embodiment is cross shaped. In alternative embodiments, the cross section of the pin may be circular, or any other suitable splined shape. The longitudinal axis of the body extends in a generally frontal-rearward (posterior-anterior) direction

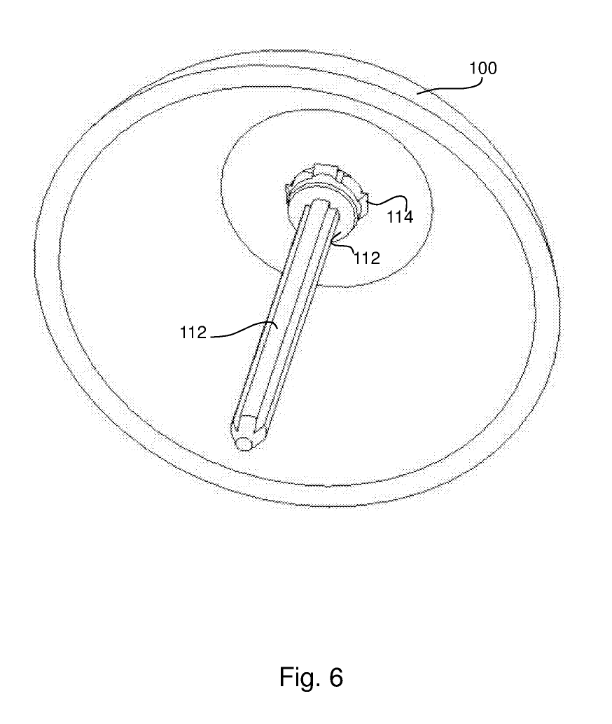

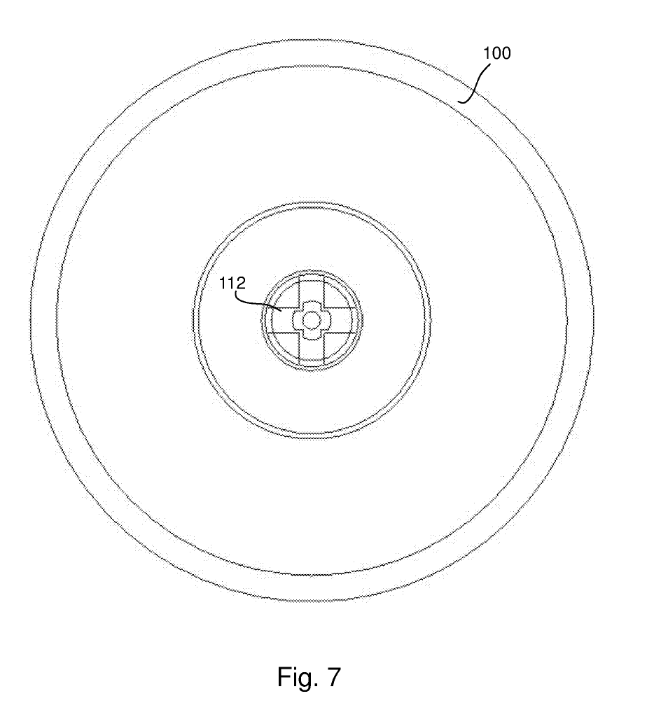

Retainer lugs 114 are positioned adjacent the head of the pin. In this embodiment four lugs 114 (only two are visible in FIG. 4) are provided, any suitable number of lugs may be used. In this embodiment, the lugs 114 protrude by approximately 1 mm. The lugs 114 are ramped towards the head 110, so as to permit the pin 108 to be pushed through the hole 104 from a convex side 116 of the substrate to a concave side 118 of the substrate 100. The axial distance between the head 110 and the legs 114 is substantially the same as the thickness of the substrate surrounding the hole 104. When the pin 108 is correctly inserted through the substrate 100, as shown in FIGS. 5 to 7, the angle of the legs 114 anchors the substrate 100 between the legs 114 and the head 110 of the pin 108, so as to prevent removal of the pin from the substrate and also restrict movement of the pin relative to the substrate.

A disc 120 is positioned along the longitudinal length of the pin 108 near the head 110 of the pin.

In this embodiment the pin 108 is manufactured from PMMA or acrylic (for example medical grade acrylic) and is a separate component to the substrate 100. In alternative embodiments the pin may be formed integrally with the substrate, e.g. by a suitable additive manufacturing/3D printing method. In further alternative embodiments the pin may be connected to the substrate using an alternative connection to the push fit and arm arrangement described, for example a bayonet type connection may be used.

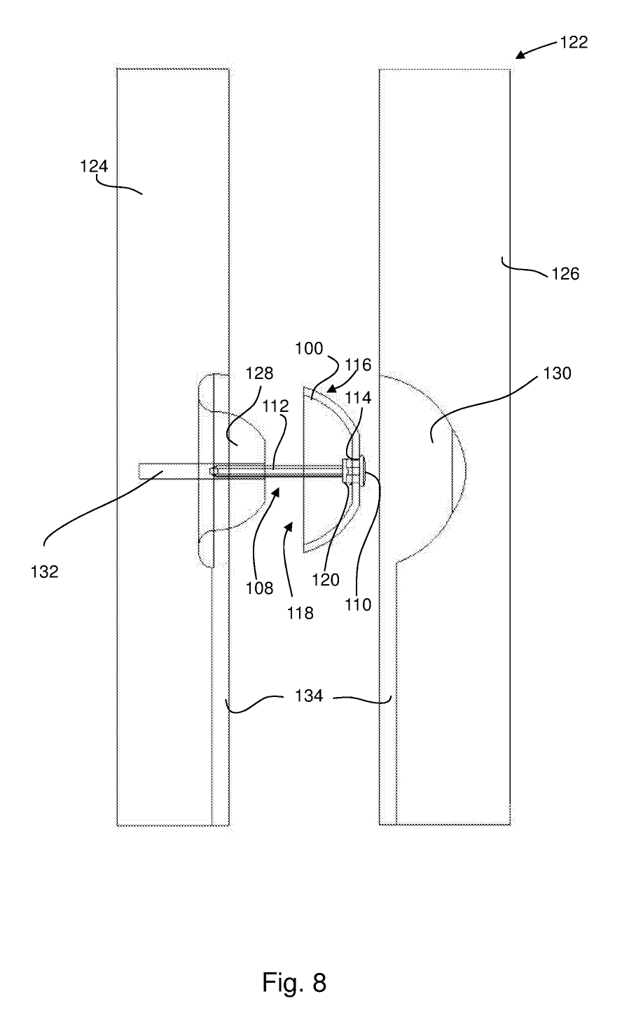

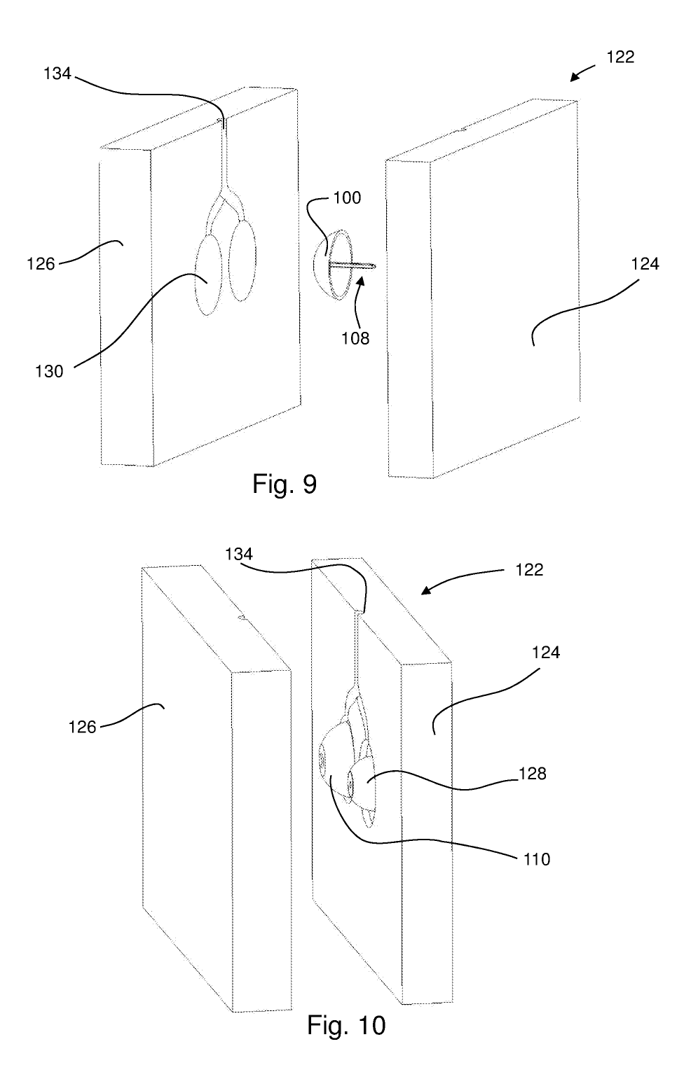

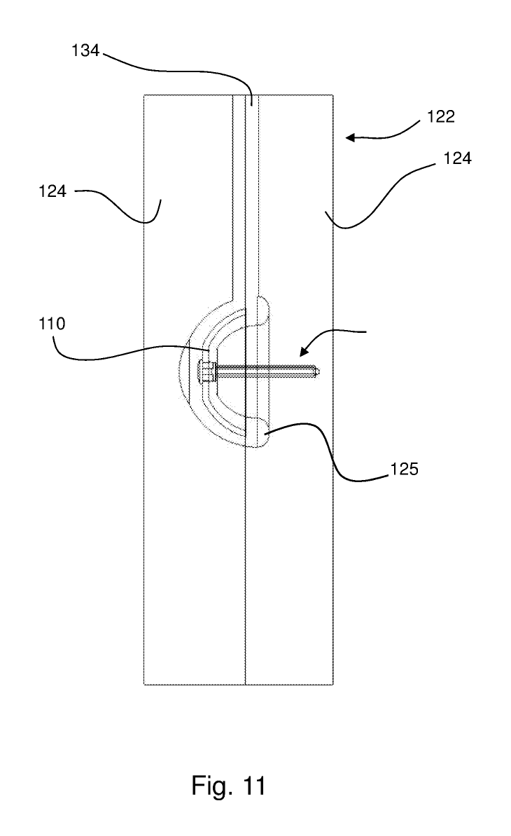

Next in step S232 the substrate 100 and pin 108 assembly is located in a mould. Referring to FIGS. 8 to 11, the mould 122 is provided in two parts, a first part 124 having a convex portion 128 and a second part 126 having a concave portion 130 defining a void 125 between them. A blind hole 132 is formed in the first part 124 extending from an apex of the convex portion into a body of the mould. The pin 108 is received in the hole 132 so as to position the concave side 118 of the substrate 100 over the convex portion 128 of the first part 124 of the mould 122. The pin 108 anchors the substrate 100 to the mould 122 so that the substrate can not move during the moulding process.

The hole 132 and pin 108 are dimensioned and positioned to ensure that the substrate 100 is located correctly from the convex 128 portion of the mould 122 within the void. In this embodiment, in the correct position, the disc 120 of the pin 108 is abutting the convex portion of the mould to provide the correct spacing in a frontal-rearward direction. The concave portion 130 of the second part 126 of the mould 122 is then positioned over the convex side 116 of the substrate 100. The dimensions of the second part 130 of the mould 122 are such that the concave portion 130 of the mould 122 is spaced from the substrate 100.

A sprue 134 is provided in the first 124 and second 126 parts of the mould 122 for supplying mould material to encapsulate the substrate (and pin). In this embodiment the mould 122 has two voids 125 to receive two separate substrates 100 and in FIG. 10 the substrate is shown located in one of the voids only. In normal use a substrate 100 would be placed in both the voids 125.

It can be seen that in this embodiment the split line between the mould parts 124 and 126 is substantially aligned with an inner (anterior) edge of a correctly positioned substrate 100 as this avoids undercuts and enables the parts to be separated when the encapsulation is complete. Advantageously, the sprue 134 is also substantially aligned with this inner edge. This eases mould separation, and minimises pressure on the pin during injection moulding, to reduce the risk of the pin fracturing. Further, this location facilitates easy removal of the solidified material in the sprue 134, and subsequent polishing. In other embodiments, especially ones where the substrate 100 does not extend to be a full hemisphere, the split line may be further towards the inner side of the void 125 than the inner edge of the substrate, so as to avoid undercuts.

In other embodiments there may be any number of voids for receiving multiple substrates, together with a suitable feeder system.

The next step S234 is to encapsulate the substrate 100 in the mould material. In this embodiment the moulding process is injection moulding. It is preferred that the split line is orientated vertically during injection moulding, as tooling may be simpler, but it can be horizontal or in any other orientation.

The mould material is PMMA or acrylic (for example medical grade acrylic) which is pumped into the voids 125 via sprue 134, but any suitable mould material may be used. In some embodiments the substrate 100 may be infiltrated with cyanoacrylate or epoxy resin; cyanoacrylate has been found to work well.

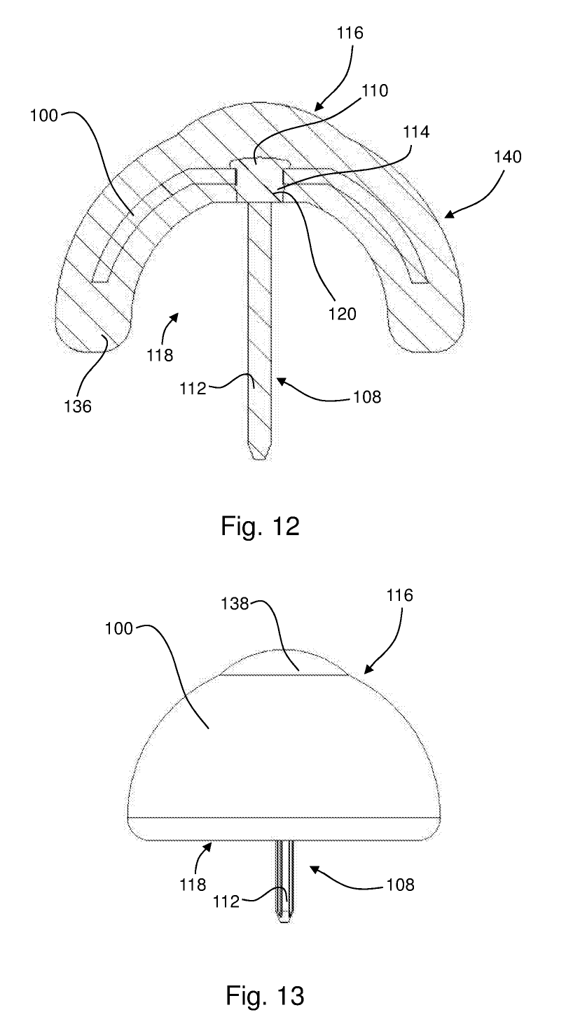

After being allowed to solidify, the encapsulated substrate 100 and pin 108 are then removed from the mould 122. FIGS. 12 and 13 show the substrate 100 and pin 108 encapsulated in the mould material 136. The spacing provided by the pin 108 and mould 122 arrangement enables the mould material 136 to surround and encapsulate the substrate

In this embodiment, the mould material 136 surrounding the substrate 100 is two to three millimetres thick. On the convex side 116 of the encapsulated substrate, a dome shape 138 is formed at an apex of the convex side.

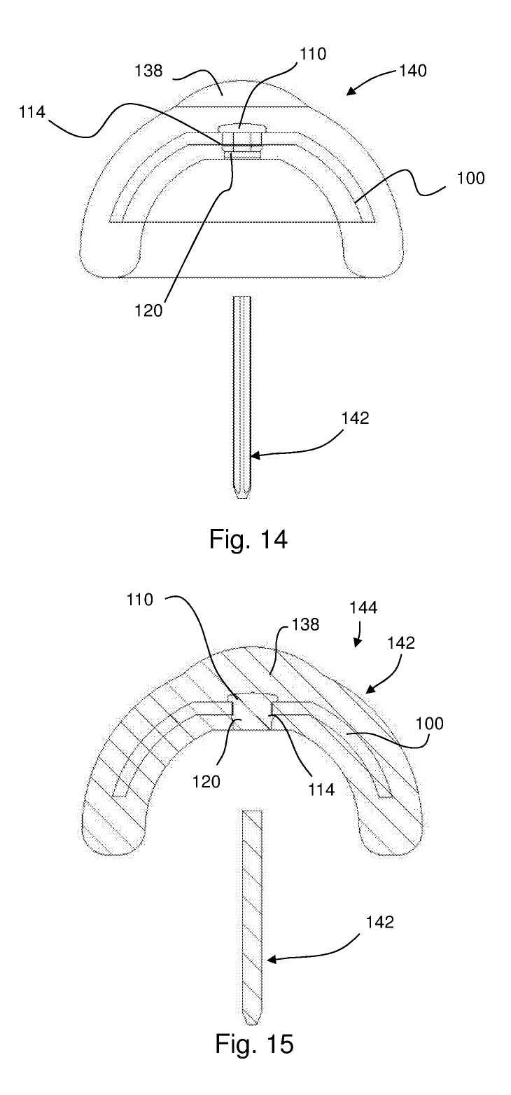

It can be seen in FIGS. 12 and 14 that the disc 120 of the pin 108 is flush with the mould material 136 on the concave side 118 of the encapsulated substrate 140. In this embodiment the pin 108 is manufactured from the same material as the mould material 136. This means that during the moulding process the pin 108 melts at the same temperature as the mould material so as to form a seal around the substrate, completely encasing the substrate, thus permitting the substrate to be fully encapsulated in a single step moulding process.

In step S236 an end of the pin 108 on the concave side 118 of the encapsulated substrate 140 is removed. Referring to FIGS. 14 and 15 in this embodiment an end 142 of the pin 108 is snapped off, but in alternative embodiments the pin 108 may be cut or melted off. The provision of the disc 120 on the length of the pin 108 and the cross section of the longitudinal body 112 enables the end 142 of the pin to be easily snapped off. In alternative embodiments the pin may remain in place depending on the intended application, for example the pin or portion thereof may be used for attachment to a body to aid positioning in an eye. The pin 108 may be weakened at the desired snapping off location in order to ease snapping off of a portion of the pin.

Finally, and optionally, in steps 238 the encapsulated substrate is polished as required to finish the artificial eye. For example, the eye may be polished at a position where the end of the pin was removed.

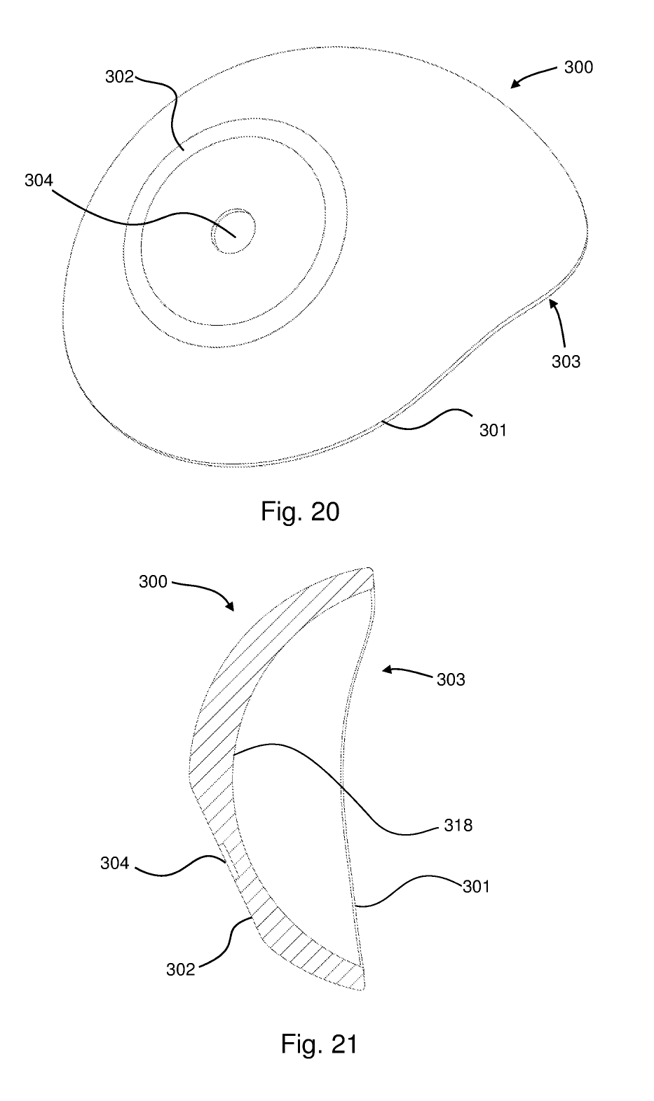

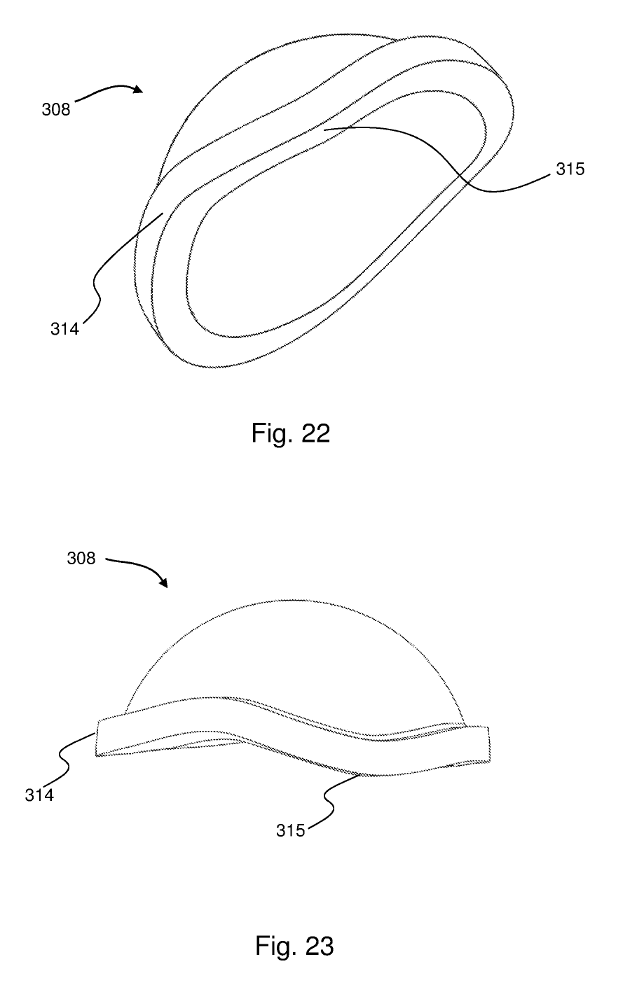

A second encapsulation method and apparatus according to the present invention is illustrated with reference to FIGS. 20 to 28. In this embodiment similar components are labelled by like numerals, but with the prefix “3”. Only differences from the first embodiment are discussed in detail.

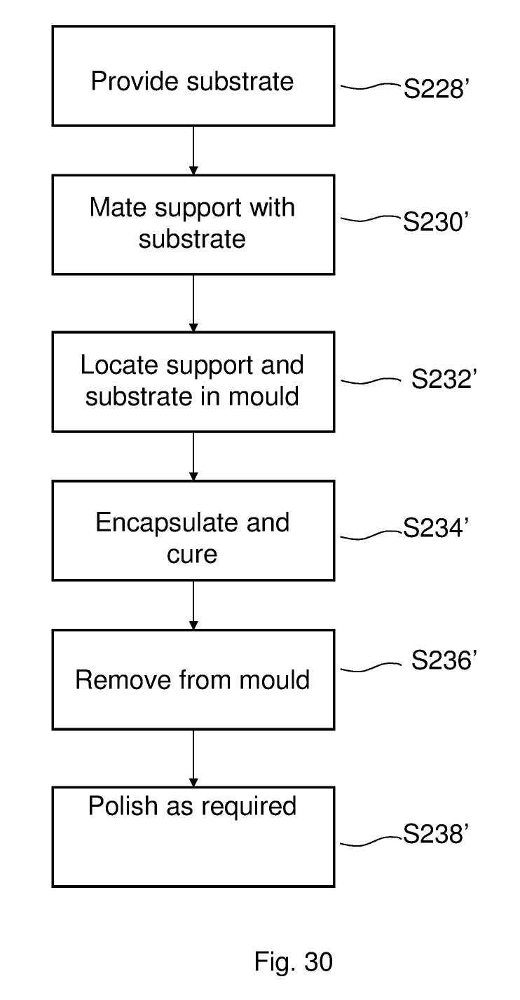

A first step S228′ of the method as illustrated by a flowchart of FIG. 30 comprises providing a substrate 300. The substrate 300 can be formed via known methods, or alternatively, as discussed below the substrate may be formed using a method of 3D printing or dye sublimation.

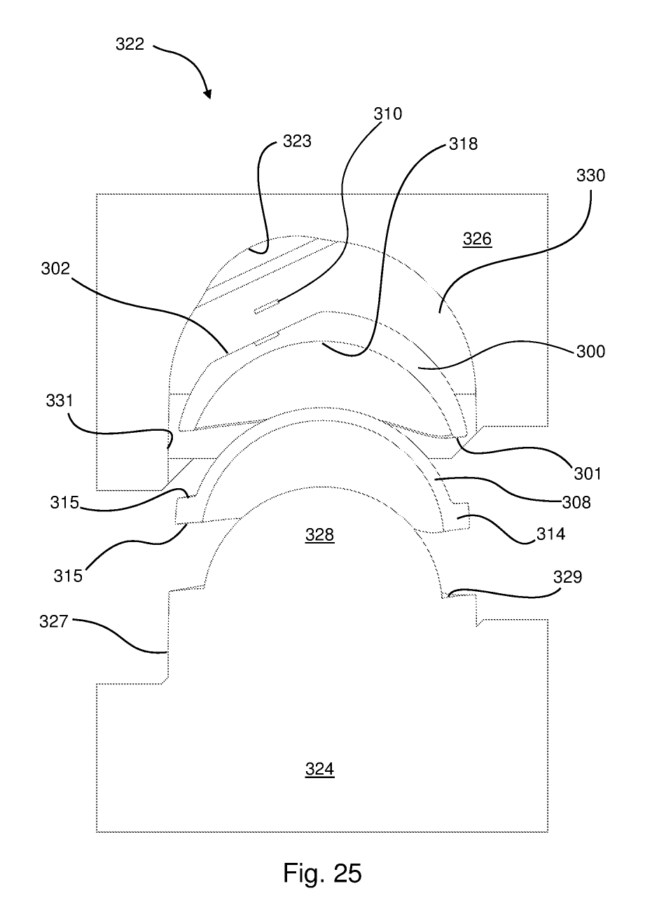

A substrate 300 is shown in FIGS. 20 and 21 and is of the type described below formed using a 3D printing method. In this embodiment the substrate has a convex shape, specifically a hollow domed shape. Offset to one side of the apex of the dome (i.e. off-centre) is a flattened region 302 upon which the iris (not shown) of the artificial eye is provided. A depression 304 is formed in the centre of the flattened region 302. A black disc, e.g. of vinyl material 310 (see FIG. 25) to represent a pupil is glued into the depression. Suitable pupils are supplied by Orbital Prosthetic Supplies of Crawley, West Sussex, UK. In other embodiments such a separate pupil may not be required if the manufacturing process for the substrate is capable of providing sufficiently dark black pupils, and this may also obviate the need for the depression 304.

In this embodiment, the substrate is approximately 1 mm thick. In embodiments where the substrate is formed by the 3D printing process described below, the raw 3D printed material is infiltrated with an acrylic prior to steps below in a dipping and baking process for 40 minutes at 100° C. In this embodiment a two-part heat cure polymer and monomer mixture is used for infiltration. Specifically, one part of J-600 PMMA polymer supplied by Factor II, Inc of Lakeside, Ariz., USA are used with ten parts of J-580-8 Non-Crosslinked Monomer, also supplied by Factor II, Inc.

An orientation feature is formed on the substrate 300. In this embodiment, the orientation feature is in the form of curved regions on a free edge 301 of the dome in diametrically opposed locations. In other embodiments other suitable steps, ridges, lugs or the like may be used. Preferably these may be positioned on the free edge 301 or concave face of the substrate 300.

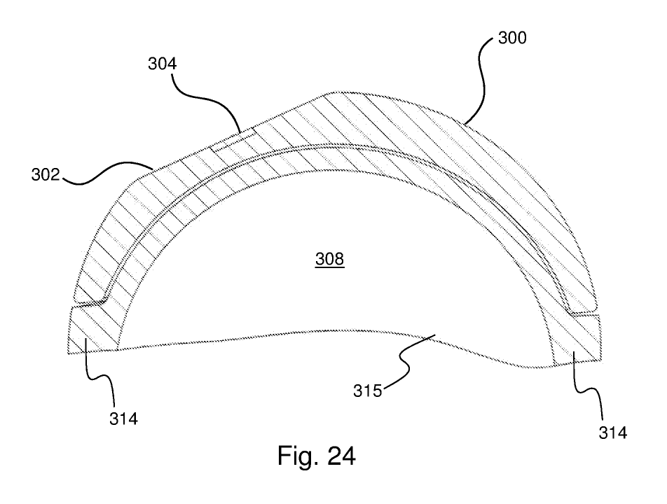

In a next step S230′ a support 308 as illustrated in FIGS. 23 and 24 in the form of a complementary domed shell is mated with the substrate. The support 308 has a lip 314 around the perimeter free edge thereof. The lip 314 has a curved profile 315 on both the top and underside thereof to act as a support orientation feature. The top of the lip 314 is arranged so as to mate in only one angular position with the substrate 300. In this position, the domed portion of the support conforms to and supports the concave underside 318 of the substrate 300. Of course, if the substrate 300 is provided with a different orientation feature, the support 308 is provided with a suitable complementary support orientation feature.

In this embodiment the support 308 if formed by injection moulding (typically transparent) PMMA or acrylic (for example medical grade acrylic) and is a separate component to the substrate 300.

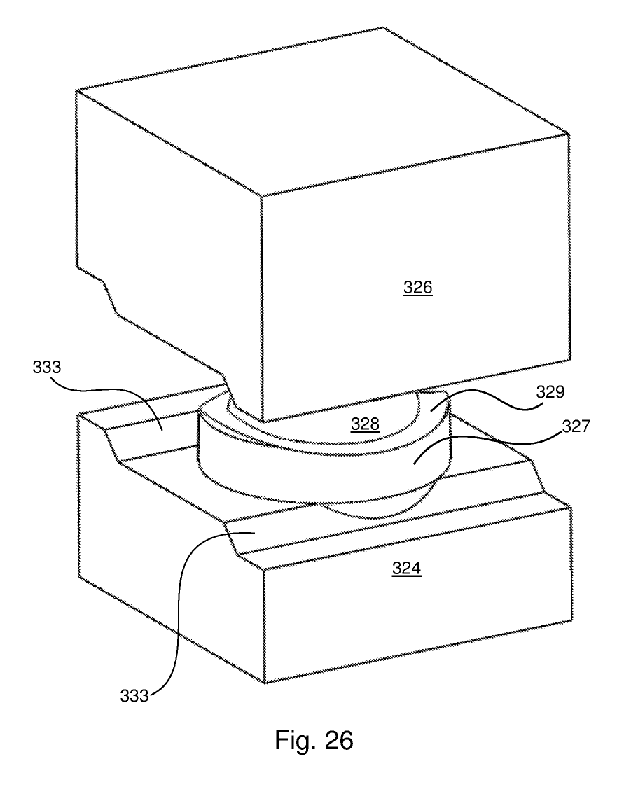

Next, in step S232′ the substrate 300 and support 308 assembly is located in a mould 322. Referring to FIGS. 25 and 26, the mould 322 is provided in two parts, a first male part 324 having a cylindrical portion 327 upon which a convex portion 328 is provided. A second female part 326 has a concave portion 330 merging into a cylindrical mouth 331 which is a close fit with the cylindrical portion 327. The convex portion 328 and concave portion 330 define a void 325 between them. The mould 322 is manufactured from stainless steel or another similar suitable material.

The convex portion 328 is dimensioned to receive the support and a base surface 329 is provided between the concave portion and the cylindrical portion 327 which undulates to align with the curved profile 315 on the support 308. Thus, the support 308 (and therefore the substrate 300) is only able to seat properly in one angular location on the first part 324 of the mould 322. In addition, the support 308 anchors the substrate 300 to the mould 322 so that the substrate is restrained from movement during the moulding process.

The concave portion 330 of the second part 326 of the mould 322 is generally domed, but formed with a with an additional off-centre depression 323 to define a shape to mimic that of a cornea on a human eye. In order to minimise post-processing, it is preferred that the concave portion 330 is polished to a smooth mirror-finish.

The mould parts 324 and 326 have complementary steps 333 on their mating faces such that the concave and convex portions are only able to fit together in one angular orientation. As a result of the orientation features on each of the aforesaid components of this embodiment, it is ensured that the off-centre flattened region 302 is correctly aligned with the depression for the cornea.

In order to encapsulate the substrate in step S236′, before closing the mould 322, the concave portion is filled with a pre-determined amount of a PMMA (acrylic) monomer in gel or putty-like form and the mould is closed and held together under suitable pressure. In this embodiment the same monomer and polymer described above for infiltration are mixed, but in a ratio of three parts polymer to one part monomer.

To cure the gel and bond it to the support 308 and the substrate 300, the mould 322 is then subjected to a heating process. In this embodiment a water bath is used. The following process is followed: 1) boil water and take off boil, 2) immerse mould in bath for 20 minutes, bring water back to boil over a period of 20 minutes, 3) boil water for 20 minutes, 4) remove mould from bath.

It can be seen that in this embodiment the split line between the mould parts 324 and 326 is also substantially aligned with an inner (anterior) edge of a correctly positioned substrate

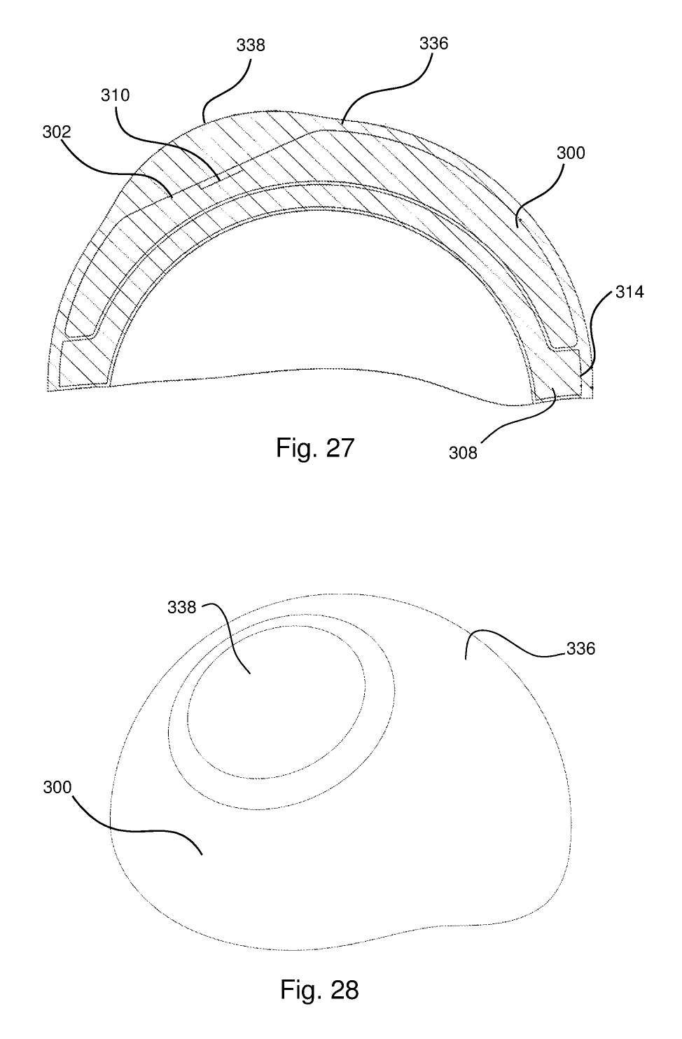

The substrate 300 which has been encapsulated by the support 308 and the cured gel mould material is then removed from the mould 322 in step S236′ and can be seen in FIGS. 27 and 28. The spacing provided by the support 308 and mould 322 arrangement enables the mould material 336 to surround and encapsulate the substrate 300.

In this embodiment, the mould material 336 surrounding the substrate 300 is two to three millimetres thick. On the convex side 316 of the encapsulated substrate, a dome shape 338 is formed off-centre to provide the cornea.

As the support 308 is manufactured from the same material as the mould material 336, the two fuse to form a good bond at the join and create a seal around the substrate 300, completely encasing the substrate. This results in the substrate being fully encapsulated in a single step moulding process.

Finally, and optionally, in step S238′ the encapsulated substrate is polished as required to finish the artificial eye.

The off-centre location of the iris may be preferable provided by the method of this embodiment may be preferable because it permits the artificial eye to be trimmed at one side only during the fitting to a patient, which may save time.

In other embodiments there may be any number of voids in each mould tool in an array for receiving multiple substrates in order to speed up the manufacturing process. The moulds may have a heating system built in to control curing. Further, the gel moulding process may be replaced by injection moulding, in a similar way to the first embodiment, although the sprue may be generally aligned with the pupil of each substrate to minimise the distance the liquid mould material has to flow.

It is preferred that the liquid or gel mould material is provided on the convex face, since the contact of this with the colours on this face that represent features of the eye has the effect of enhancing the vividness of the colours. However in other embodiments, in particular where a different type of substrate is used, it is anticipated that the support may be provided on the convex face.

It will be further appreciated that the substrate, support and pupil may be assembled in a different order to that described above. The support may not cover the entire concave (or convex) face of the substrate—for example certain portions may be cut away and addition gel monomer provided to complete the encapsulation.

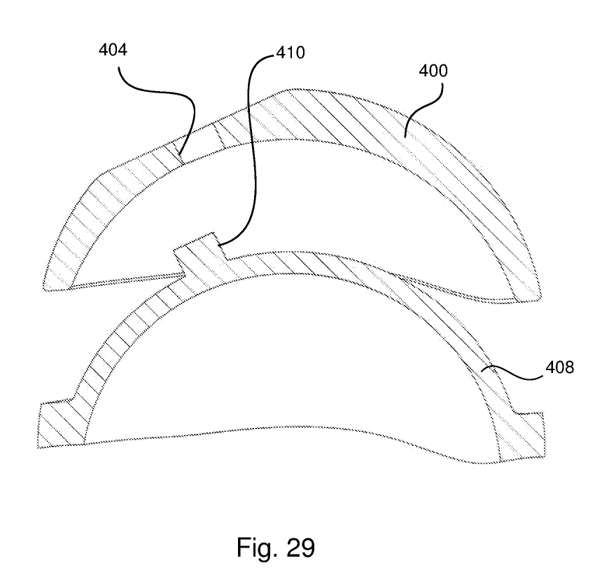

In a further variant shown in FIG. 29, a hole 404 is provided in the substrate 400 in the centre of the iris and the support 408 has a protrusion 410 shaped to protrude therethrough. At least this portion of the support is coloured black, either by utilising a “two shot” injection moulding process in its manufacture, in which the black portion is moulded on top of the rest of the support in a second injection moulding step, or the entire support is coloured black with suitable pigment, thereby removing the need for a separate disc to represent the pupil. In other respects this variant and its manufacturing method is the same as the second embodiment, however.

The above described method of manufacture produces an artificial eye that is fully encapsulated in a mould material using a single step moulding process. This is advantageous over manufacturing processes of the prior art that generally require at least a two step moulding process to fully encapsulate the substrate 100 in mould material.

An artificial eye manufactured from the substrate 100 may be required as a bespoke or a stock eye. Considering a stock eye the manufacturing process is primarily to be used for the production of stock or “off the shelf” eyes that may be produced in a range of standard sizes and colours, and which may be used as a temporary artificial eye, or a lower cost permanent eye, e.g. to be used in developing countries. In addition to the entirely off the shelf approach, a standard sized and shaped prosthesis may be used in conjunction with an image which has been matched to a particular patient by an acquisition process set out below.

The artificial eye may be of the type intended to be non-integrated, or of the type intended to be integrated e.g. an orbital implant.

Now the process of forming the substrate for an artificial eye using a method of 3D printing or dye sublimation will be described.

With reference to in particular FIG. 17, manufacturing methods according to two embodiments of the invention are illustrated, in which certain steps are common, and other steps are not as described below.

The method of both embodiments of the present invention commences with the acquisition of an image of the visible portion of an existing eye at step S200. This image is preferably taken using a high quality digital camera such as a single lens reflex (SLR) camera. The image may be of a particular patient's eye before being replaced, may be of a patient's other eye that is not being replaced (if the eye to be replaced is injured to the extent that an image may not be acquired), or if the artificial eye is to be a “stock” eye it may simply be of any person's eye in order to be used with a collection that is representative of a number of different general eye colours and sizes for subsequent “off the shelf” use.

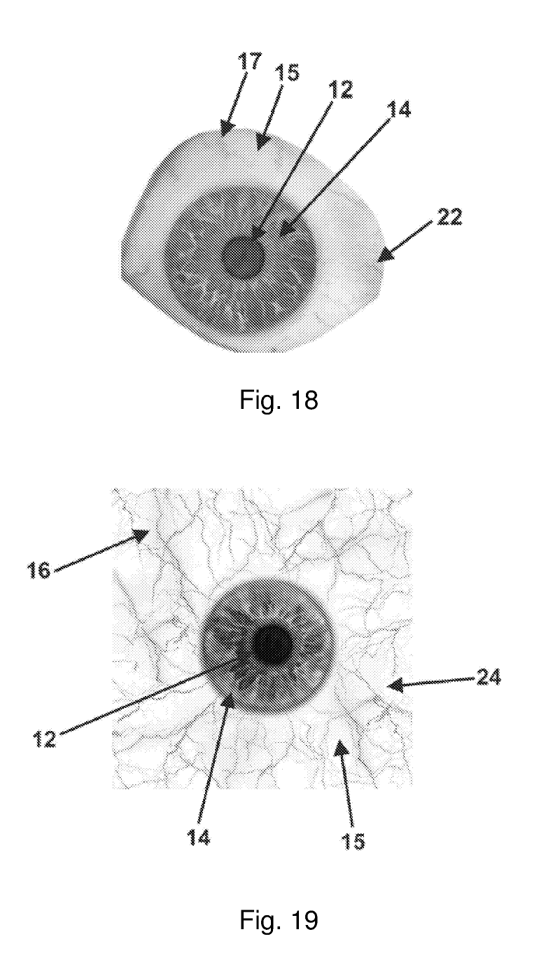

With reference to FIG. 18 the image 22 that is acquired is incomplete since only a portion of the eye in situ can be made visible at any time. The image 22 comprises a pupil 12, iris 14, and sclera 15 having a particular pattern of veins 17 visible thereon. At step S202 the image is edited using suitable photo manipulation software such as Adobe Photoshop®. At this stage, the iris 14 and pupil 12 are separated from the remainder of the image and the image is colour corrected to remove a colour cast that may be present due to the lighting conditions under which the photograph is acquired. In addition, the image 22 is adjusted to account for the particular colour profile of the printer upon which the image will be output. In particular, the image is acquired and stored as an RGB image whereas the printer prints using a CYMK colour palette and appropriate corrections need to be made.

At step S204 simulated veins 16 are applied to the image and the image canvas is extended to a sufficient area for subsequent coverage of an artificial eye blank to provide a manipulated image 24 as illustrated in FIG. 19. In particular, a suitable Photoshop brush may be used to apply the simulated veins. In a variant of the process the veins 17 from the acquired image may be retained, and simulated veins 16 may be matched thereto for the remainder of the canvas.

At step S206 the manipulated image 24 is scaled to an appropriate size. In selecting the size, it is preferable that consideration is made for the apparent enlargement of the features of the eye that will occur as a result of optical effects caused by the subsequent encapsulation process, as well as a general desire to have the iris 14 of the artificial eye appear slightly smaller than the iris of the patient's “real” eye since this tends to draw attention away from the artificial eye and it is therefore less noticeable.

At this point, the process diverges dependent upon the type of artificial eye that is required. Considering the first or “stock” process:

At step S208 the scaled image produced at S206 is overlaid on a CAD model of the required prosthesis and is positioned at an appropriate location with a pupil region at the frontal portion of the model. In a preferred embodiment the image is also overlain on a reverse (concave) face of the model in register the same image on the convex face.

In a preferred embodiment, Magics rapid prototyping software, produced by Materialise of Leuven, Belgium is used for this stage of the process with the CAD model being in STL format. In one variant, the scaling of the image 24 is undertaken at this stage in Magics, rather than or in addition to the scaling that is undertaken at step S206 in Photoshop. A close-up image of the patient's real eye may be used at this stage to ensure that a size and position good match is achieved for the artificial eye. The Magics software may then export the finished model in a suitable CAD file format for manufacturing on a 3D printer.

At step S210 in a preferred embodiment of the present invention, a Z Corporation Spectrum model 510 3D printer is used and Magics exports the CAD file in the proprietary ZPR or ZCP format that is suitable for use with this type of printer.

3D Systems Corp of Rock Hill, S.C., USA (formerly Z Corporation) produces a range of 3D printers in the ZPrinter range that also includes models 450 and 650, which function using a proprietary process that builds up a 3D product in layers from powder material, and are also suitable for use in the method of the present invention. The printer has four print heads that “print” a binder material into powder selectively in conjunction with coloured inks (one head for each colour) to produce coloured 3 dimensional objects, in a manner akin to a standard inkjet printer. The printers have a resolution upwards of 300×300 dpi in the X and Y direction and a layer thickness of as little as 0.1 mm (Z direction). The way in which the colour is mixed in with the binder means that the colour penetrates a distance into the eye itself and is an intrinsic part of the finished eye, rather that a layer on the surface.

In this embodiment, a silica powder having the designation ZP150 and a binder having the designation B60 are used. Both are supplied by 3D Systems Corp (formerly Z Corporation). This powder is bleached to produce objects that are by default white or substantially so. The artificial eyes are preferably printed using this printer with the outermost (anterior) portion of the eye when fitted uppermost on the print bed since this produces a strong finished eye. As the print bed is substantially larger than a single artificial eye, multiple eyes can be manufactured simultaneously in an X and Y direction, and may also be stacked on top of each other in the Z direction.

In a preferred embodiment, as a result of superimposing the image 24 onto both the convex and concave faces of the CAD model the image is printed on both faces 18 and 20 of the finished 3D article, and in view of the degree of translucency of the material at this thickness, the resultant artificial eye appears to have a more vivid, realistic, colouring.

In an alternative embodiment, instead of printing the vein colour on the surface layer of powder, a predetermined depth of material (e.g. 0.2-1 mm) is coloured below the surface parallel to the frontal-to-rear (posterior-anterior) axis, as the eye is built up. This has been found to improve the colour and clarity of the veins. A similar approach is also used to improve iris colouration.

Once the printing process is complete, at step S212 the artificial eyes are removed from the bed of powder and are cleaned using a stiff brush or by sand blasting and are then air brushed with compressed air to remove any remaining dust and particles.

At step S214 the artificial eye is then immersed in a low viscosity bonding agent that is substantially colourless, for example cyanoacrylate, in this embodiment Procure PC08 produced by Cyanotech of Dudley, UK. This product has the advantage of being an approved substance for use in the manufacturing of medical devices.

Once removed from the bonding agent and when curing is complete, at step S216 the artificial eye may then be encapsulated using the previously described method.

In the second example, the manufacturing steps after S206 differ and are as follows:

At step S218 the scaled and colour corrected image is printed onto a transfer material, which in this embodiment is dye sublimation film using dye sublimation ink in an inkjet printer. A preferred ink is Artrainium ink supplied by Sawgrass of Sheffield, UK in CYMK and light cyan light magenta colours.

In this embodiment a cornea blank have the general solid domed shape of the finished artificial eye, but no colouring, is manufactured from PMMA using a known process. The blank of this embodiment is termed “bespoke” because the rear (anterior) thereof is shaped in accordance with a cast that has been taken of a particular patient's eye socket, again using a known process, and thus is specifically intended for use with that patient.

The cornea blank is then pre-coated with an adhesion promoter such as Digicoat as supplied by Octi-tech Limited of Sheffield, UK, which is then wiped off and followed by application of a sublimation coat that may also be supplied by Octi-tech Limited in the Digicoat range. This process occurs at step S220.

At step S222 the pre-treated cornea blank is then loaded at a predetermined location into a vacuum press for the dye sublimation ink to be transferred onto the blank. A suitable vacuum press for this to be achieved is a Pictaflex PF480/6 model as supplied by I-Sub of Kettering, UK. This press has a sufficiently large bed that an array of blank prostheses may be arranged at suitable locations that correspond to printed images on the sublimation film, and the transfer may then simultaneously have images transferred to them. In a preferred embodiment, a supporting grid (e.g. of sheet metal with an array of apertures provided therein) is preferably placed around the individual images to prevent the sublimation film from sagging during the image application process set out below.

In the vacuum press machine, the chamber is heated, the bed supporting the blank prosthesis is raised and a vacuum is generated in order to suck the sublimation film onto the blank. The heat causes the sublimation ink to be transferred from the film onto the prosthesis in an appropriate location. After an appropriate dwell time, the vacuum is removed and the film and blank separated and the press is allowed to cool.

Table 1 below sets out examples of various heat and dwell time parameters that have been used. Example 5 has been found to provide the best results.

TABLE 1

| | Standard | | | | | | |
|---|---|---|---|---|---|---|---|
| | Setting | Ex. 1 | Ex. 2 | Ex. 3 | Ex. 4 | Ex. 5 | Ex. 6 |
| Pre-heat temp | 120 | no pre-heat | no pre-heat | no pre-heat | no pre-heat | no pre-heat | no pre-heat |
| (° C.) | | | | | | | |
| Pre-heat time | 20 | 0c | 0 | 0 | 0 | 0 | 0 |
| (sec) | | | | | | | |
| Fill temp (° C.) | 135 | 135 | 135 | 145 | 135 | 135 | 135 |
| Vacuum time | 8 | 8 | 8 | 12 | 12 | 12 | 12 |
| (sec) | | | | | | | |
| Air temp (° C.) | 190 | 190 | 200 | 220 | 180 | 190 | 200 |
| Transfer | 170 | 180 | 190 | 200 | 160 | 175 | 185 |
| temp* (° C.) | | | | | | | |
| Print time | 150 | 160 | 170 | 180 | 160 | 120 | 120 |
| (sec) | | | | | | | |
| Release time | 15 | 15 | 15 | 20 | 15 | 5 | 5 |
| (sec) | | | | | | | |
| Cooling time | 15 | 15 | 30 | 35 | 30 | 30 | 30 |
| (sec) | | | | | | | |
| Unload | 60 | 60 | 60 | 60 | 35 | 35 | 35 |
| (sec) | | | | | | | |

*Transfer temp = ideal temperature to transfer the image from film to eye

The blank is then removed from the vacuum press at step S224 and a clear lacquer is then applied to the image that has been transferred so to minimise the bleeding of colours at step S226. A currently preferred lacquer is “Very high temperature lacquer” supplied by Hycote of Oldham, UK.

At step S216, the printed blank (or substrate) is encapsulated using the previously described method.

It will be appreciated that numerous changes may be made within the scope of the present invention. For example certain steps of the processes may be altered in their order; various steps may be taken at different times and in different locations. Other suitable 3D printers, dye sublimation materials, and vacuum presses, and image manipulation techniques and software may be used. The blanks/substrate used need not have a wholly domed shape. For example, the iris region may be substantially flat, and the finished domed shape built up from the encapsulation material.

It will be appreciated that terms such as front and rear, upper and lower are used for ease of explanation, and should not be regarded as limiting.

In order to minimise the complexity of the process, it is envisaged that for example three standard eye diameters would be used, and three different depths of substrate (in a frontal to rear direction).

Other proprietary 3D printing systems may be used to produce a suitable substrate, including, by way of example, an Iris printer supplied by MCOR technologies of Dunleer, Co. Louth, Ireland and an Objet500 Connex supplied by Stratasys Limited of Minneapolis, Minn., USA. These systems may produce a substrate in which the substrate can be coloured a deep enough black to obviate the need for a separate insert or the black being provided on the support.

It will further be appreciated that the method of the present invention is a way encapsulating or finishing in which artificial eyes at significantly less cost than prior art techniques, which means that higher quality artificial eyes may be supplied in developing countries where previously the cost would be prohibitive. The resultant artificial eyes have been found to be of at least similar or of higher quality than those produced by prior art methods.

1. A method of manufacturing an artificial eye for fitting as a whole or partial replacement of a patient's original eye, the method comprising the steps of:
a) providing an image of an iris on a substrate comprising at least a frontal region of an artificial eye;
b) providing a support for the substrate;
c) positioning the substrate and support in a mould; and
d) encapsulating the substrate within a mould material.

2. A method according to claim 1, wherein the support is a pin

3. A method according to claim 1, wherein in the support is provided on an internal side of the eye.

4. A method according to claim 1, wherein the support is provided as a separate component to the substrate.

5. (canceled)

6. A method according to claim 4, comprising the step of positioning a head of the support on a frontal side of the substrate, e.g. to form the pupil of the artificial eye.

7. A method according to claim 4, comprising the step of providing a hole in the substrate and positioning the support through the hole of the substrate.

8. A method according to claim 6, comprising the step of providing a recess in the substrate circumferentially around the hole for seating a head of the support.

9. A method according to claim 6, comprising the step of pushing the support from an inner side of the substrate through the hole towards an outer side of the substrate, wherein the method further comprises the step of providing lugs on the support positioned and arranged so as to retain the support on the substrate once it has been pushed through the hole.

10. (canceled)

11. A method according to claim 1, wherein the mould comprises a hole and the method comprises the step of locating the support in the hole of the mould.

12. A method according to claim 1, comprising the step of removing an end of the support once the substrate is encapsulated, wherein the portion of the support is snapped off once the substrate is encapsulated.

13-15. (canceled)

16. A method according to claim 1, wherein the iris is off-centre from an apex of the substrate.

17. A method according to claim 1, wherein the support is arranged to support substantially all an external or an internal face of the substrate.

18-27. (canceled)

28. A pre-moulded artificial eye comprising:
a substrate having at least a frontal region of an artificial eye;
an image of an iris positioned on an outer side of the substrate; and
a support for the substrate to suitably position the pre-moulded eye with a mould.

29. An artificial eye comprising:
a substrate layer having an image of an iris formed thereon;
a support located at an inner side of the substrate, and arranged to orientate the substrate with respect to the support; and
transparent mould material arranged to encapsulate the substrate in conjunction with the support.

30. An artificial eye according to claim 29, wherein the support is further arranged to orientate the support with respect to a mould.

31-34. (canceled)

35. A method according to claim 4, wherein an orientation feature is provided to orientate the substrate with respect to the support.

36. A method according to claim 1, wherein an orientation feature is provided to orientate the support with respect to the mould.

37. A pre-moulded artificial eye according to claim 35, wherein the support is arranged to orientate the substrate with respect to the support.

38. A pre-moulded artificial eye according to claim 35, wherein the support is further arranged to orientate the support with respect to the mould.

39. A pre-moulded artificial eye according to claim 35, wherein the iris portion may be off-centre from an apex of the substrate.
